(19) United States
(12) Patent Application Publication
(10) Pub. No.: US 2023/0022841 A1
(43) Pub. Date: Jan. 26, 2023

(54) ISARIDIN CYCLODEPSIPEPTIDE DERIVATIVES, AND PREPARATION AND APPLICATION THEREOF

(71) Applicant: Sun Yat-sen University, Guangzhou (CN)

(72) Inventors: Senhua CHEN, Zhuhai (CN); Lan LIU, Zhuhai (CN); Minghua JIANG, Zhuhai (CN)

(21) Appl. No.: 17/899,161

(22) Filed: Aug. 30, 2022

Related U.S. Application Data
(63) Continuation of application No. PCT/CN2022/114544, filed on Aug. 24, 2022.

(30) Foreign Application Priority Data
Jan. 10, 2022 (CN) 202210019737.2

(51) Int. Cl. C07K 11/02; C07K 1/20; C07K 1/14; A61P 7/02

(52) U.S. Cl. CPC C07K 11/02; C07K 1/20; C07K 1/145; A61P 7/02; A61K 38/00

(57) ABSTRACT

Provided herein are an isaridin cyclodepsipeptide derivative, and a preparation and an application thereof. The isaridin cyclodepsipeptide derivative is shown in formula (I), and is isolated from ascidian-associated fungi.

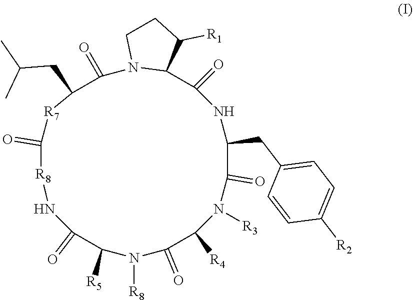

### CROSS-REFERENCE TO RELATED APPLICATIONS

This application is a continuation of International Patent Application No. PCT/CN2022/114544, filed on Aug. 24, 2022, which claims the benefit of priority from Chinese Patent Application No. 202210019737.2, filed on Jan. 10, 2022. The content of the aforementioned applications, including any intervening amendments thereto, is incorporated herein by reference in its entirety.

### TECHNICAL FIELD

This application relates to biomedicine, in particular to a type of isaridin cyclodepsipeptide derivatives, and preparation and application thereof.

### BACKGROUND

The thrombotic disease is a dangerous pathological process posing a major threat to human health and life owing to its high morbidity and mortality. Thrombosis will be developed into various cardiovascular diseases, including coronary atherosclerotic heart disease, myocardial infarction, ischemic stroke and pulmonary embolism.

Currently, the clinical anti-thrombotic drugs mainly include anticoagulants and anti-platelet drugs, which can directly inhibit the formation of thrombosis to treat thrombotic diseases. However, most of drugs, such as warfarin and aspirin, are often accompanied by serious adverse effects and poor therapeutic effects. Those skilled in the art have also developed many compounds which were demonstrated to have anti-thrombotic activity. For example, Chinese Patent Publication No. 1228701A discloses a heterocyclic compound with thrombolytic activity. This compound exhibits a certain anti-thrombotic effect but has no obvious effect on thromboinflammation, which is a key risk factor for venous occlusion and thrombus death and can easily lead to organ necrosis. In addition, this compound is not suitable for industrial production due to complicated preparation and high cost. Given the defects in the prior art, it is urgently needed to develop a new anti-thrombotic drug to promote the clinical treatment of thrombosis-related diseases.

### SUMMARY

In order to overcome the defects that the existing anti-thrombotic drugs have adverse reactions, no anti-inflammatory effect and poor therapeutical effect, and struggle with complicated preparation and high preparation cost, this application provides a type of isaridin cyclodepsipeptide derivatives with significant anti-inflammatory and anti-thrombotic activities and excellent safety and reliability.

Another object of this application is to provide a method for preparing the isaridin cyclodepsipeptide derivatives.

Another object of this application is to provide an application of the isaridin cyclodepsipeptide derivatives.

Another object of this application is to provide a type of anti-inflammatory and/or anti-thrombotic drugs.

The technical solutions of the present disclosure are described as follows.

In a first aspect, this application provides a isaridin cyclodepsipeptide derivative of formula (I), or a pharmaceutically acceptable salt, ester or solvate thereof:

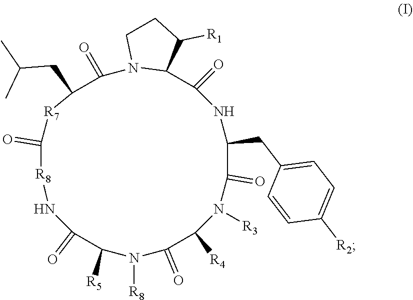

wherein R₁ is —H or —CH₃; R₂ is —H or —OH; R₃ and R₈ are each independently —H or —CH₃; R₄ and R₅ are each independently selected from the group consisting of —CH₃, —CH₂CH₃, —CH(CH₃)₂, —CH₂CH(CH₃)₂, —CH(CH₃)CH₂CH₃, —CH(OH)CH₃ and —CH₂Ar; R₆ is —CH₂— or —CH₂CH₂—; and R₇ is —NH— or —O—.

In an embodiment, the isaridin cyclodepsipeptide derivative comprises one 2-hydroxy-4-methylvaleric acid or its analog Leucine, one Proline or 3-methyl-proline, one Phenylalanine or Tyrosine, 0˜2 N-methylated amino acids, and one β-Alanine or Glycine.

In an embodiment, the isaridin cyclodepsipeptide derivative is selected from the group consisting of:

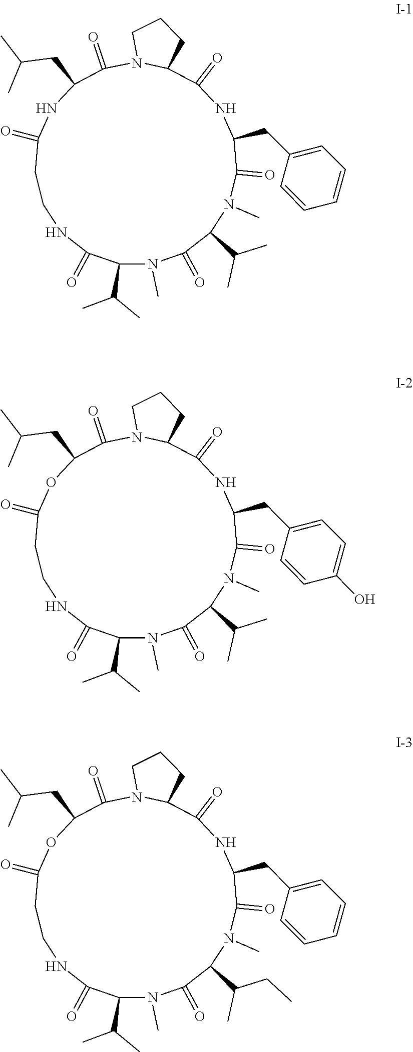 [SMILES: CC(C)C[C@@H]1NC(=O)CCNC(=O)[C@H](C(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)N(C)C(=O)[C@H](Cc2ccc(O)cc2)NC(=O)[C@@H]2CCCN2C1=O ; CCC(C)[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C]

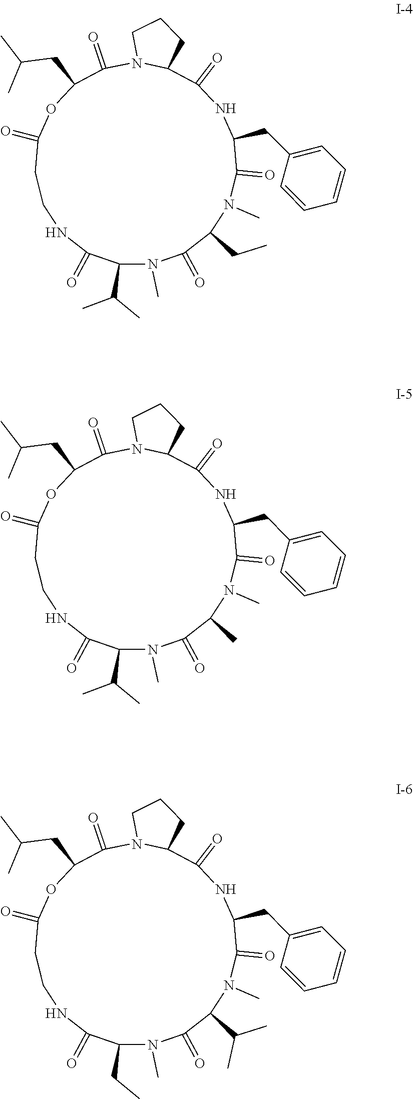 [SMILES: CC[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C ; CC(C)C[C@@H]1OC(=O)CCNC(=O)[C@H](C(C)C)N(C)C(=O)[C@H](C)N(C)C(=O)[C@H](Cc2ccccc2)NC(=O)[C@@H]2CCCN2C1=O ; CC[C@H]1C(=O)NCCC(=O)O[C@@H](CC(C)C)C(=O)N2CCC[C@H]2C(=O)N[C@@H](Cc2ccccc2)C(=O)N(C)[C@@H](C(C)C)C(=O)N1C]

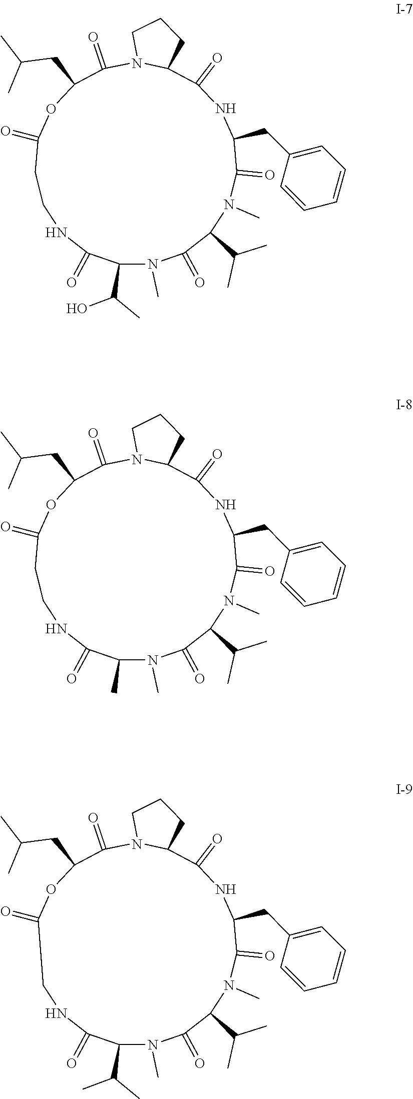 [SMILES: CC(C)C[C@@H]1OC(=O)CCNC(=O)[C@H](C(C)O)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CNC(=O)[C@H](C(C)C)N(C)C(=O)[C@H](C(C)C)N(C)C(=O)[C@H](Cc2ccccc2)NC(=O)[C@@H]2CCCN2C1=O]

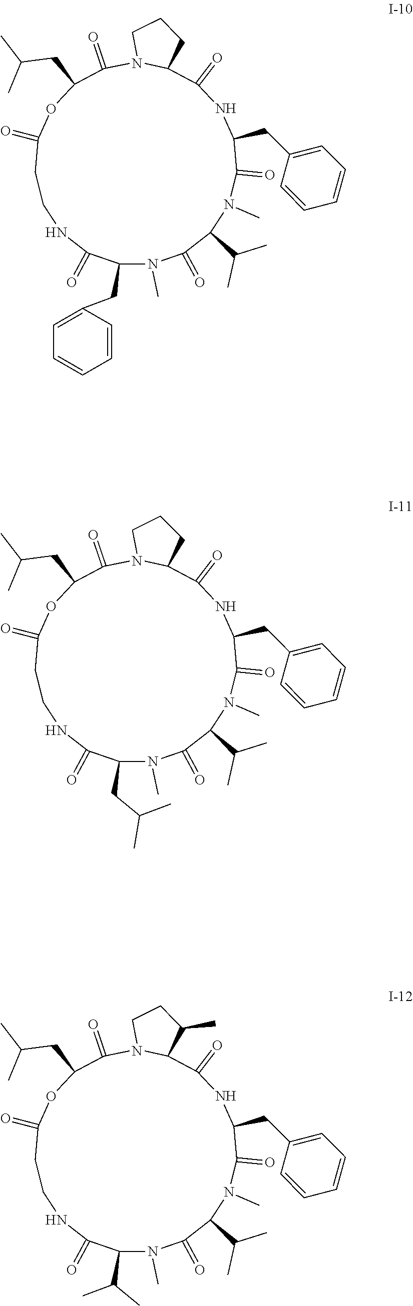 [SMILES: CC(C)C[C@@H]1OC(=O)CCNC(=O)[C@H](Cc2ccccc2)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](CC(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)N(C)C(=O)[C@H](Cc2ccccc2)NC(=O)[C@@H]2[C@H](C)CCN2C1=O]

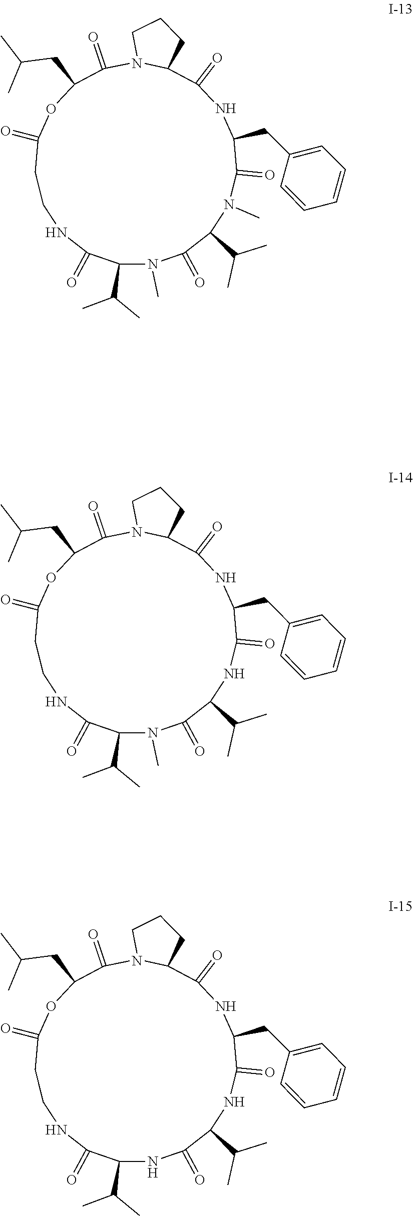 [SMILES: CC(C)C[C@@H]1OC(=O)CCNC(=O)[C@H](C(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)NC(=O)[C@H](Cc2ccccc2)NC(=O)[C@@H]2CCCN2C1=O ; CC(C)C[C@@H]1OC(=O)CCNC(=O)[C@H](C(C)C)NC(=O)[C@H](C(C)C)NC(=O)[C@H](Cc2ccccc2)NC(=O)[C@@H]2CCCN2C1=O]

In an embodiment, a pharmaceutically-acceptable salt of the isaridin cyclodepsipeptide derivative is expressed as formula (II):

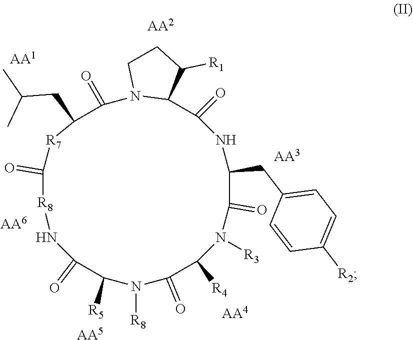

wherein AA¹ is Leucine or 2-hydroxy-4-methylvaleric acid; AA² is Proline or 3-methylproline; AA³ is Phenylalanine or Tyrosine; AA⁴ and AA⁵ are independently selected from the group consisting of Alanine, 2-Aminobutyric acid, Valine, Isoleucine, Leucine, Threonine, Phenylalanine, N-methylated Phenylalanine derivative and a combination thereof; AA⁶ is Glycine or β-Alanine.

In a second aspect, this application provides a method for preparing the isaridin cyclodepsipeptide derivative, comprising:

preparing the isaridin cyclodepsipeptide derivative from Beauveria felina SYSU-MS7908 by isolation and purification;

wherein the Beauveria felina SYSU-MS7908 has been deposited in Guangdong Microbial Culture Collection Center (GDMCC) (No. 100, Xianlie Middle Road, Yuexiu District, Guangzhou, Guangdong Province, China) on Jul. 24, 2020, and has a deposit number of GDMCC No: 61059.

In an embodiment, the isaridin cyclodepsipeptide derivative is prepared through steps of:

(1) subjecting the Beauveria felina SYSU-MS7908 to enlarged culture to collect a fungal fermentation broth extract by an organic solvent and

(2) subjecting the fungal fermentation broth extract to liquid separation extraction, concentration, silica gel column chromatography, Sephadex LH-20 column chromatography and reversed-phase high-performance liquid chromatography (RP-HPLC) to obtain the isaridin cyclodepsipeptide derivative.

In an embodiment, in step (1), the organic solvent is selected from the group consisting of acetone, ethyl acetate, methanol and ethanol.

In an embodiment, in step (2), the extraction is performed in ethyl acetate, chloroform or a combination thereof.

Specifically, the method for preparing the isaridin cyclodepsipeptide derivatives comprises:

(S1) inoculating the Beauveria felina SYSU-MS7908 into a seed medium followed by shaking or static culture to obtain a seed liquid, and transferring the seed liquid into a fermentation medium for fermentation to collect the fungal fermentation broth;

(S2) subjecting the fungal fermentation broth to extraction 2˜5 times with an organic solvent and concentration to obtain the extracts;

(S3) subjecting the extract to extraction 2˜5 times and concentration to obtain a concentrate; and subjecting the concentrate to gradient elution on a silica gel column with a series of ethyl acetate-petroleum ether solutions respectively having a volume ratio of 10%, 20%, 30%, 45%, 60% and 100% and methanol-ethyl acetate solutions having a volume ratio of 5% and 10% to obtain 8 fractions Fr. A-Fr. H;

(S4) collecting fractions Fr. C and Fr. D eluted respectively by 30% and 45% ethyl acetate-petroleum ether solutions;

subjecting the fractions Fr. C and Fr. D to reverse-phase silica gel column chromatography with an elution gradient of 30%, 50%, 70% and 90% methanol/water solutions;

collecting 50-90% fractions followed by dissolving in hot ethanol and recrystallization to obtain compound I-13; and

collecting remaining mother liquors Fr. C-L1 and Fr. D-L1;

(S5) passing the mother liquor Fr. C-L1 through Sephadex LH-20 (a volume ratio of dichloromethane to methanol is 1:1) followed by purification via RP-HPLC (isocratic elution), wherein a mobile phase is 45-65% methanol/water or 30-50% acetonitrile/water; and

collecting eluted fractions with a retention time of 10˜30 min to obtain some isaridin cyclodepsipeptide derivatives: compounds I-14, I-4, I-5, I-6, I-8 and I-9;

(S6) passing the mother liquor of Fr. D-L1 through Sephadex LH-20 (V_{dichloromethane}/Vₘₑₜₕₐₙₒₗ 1:1) followed by purification via RP-HPLC (isocratic elution), wherein a mobile phase is 45˜65% methanol/water or 30˜50% acetonitrile/water; and

collecting eluted fractions with a retention time of 10˜30 min to obtain some isaridin cyclodepsipeptide derivatives: compounds I-1, I-3, I-10 and I-11; and

(S7) passing the fraction Fr. E (eluted by 60% ethyl acetate-petroleum ether solution) through Sephadex LH-20 (methanol) followed by reverse-phase silica gel column chromatography using gradient elution with 30%, 50%, 70% and 90% methanol/water solutions;

collecting a fraction eluted by the 70% methanol/water solution followed by RP-HPLC (isocratic elution), wherein a mobile phase is 45˜65% methanol/water or 30%˜50% acetonitrile/water; and

collecting an eluted fraction with a retention time of 10˜30 min to obtain compounds I-12, I-15, I-2 and I-7.

In an embodiment, in step (S1), the seed medium is a commercially-available potato dextrose broth (containing 200 g/L of potato and 20 g/L of glucose), a yeast extract-peptone-dextrose (YPD) liquid medium (containing 50 g/L of peptone, 20 g/L of yeast extract and 4 g/L of glucose) or a yeast extract-peptone-dextrose agar medium (containing 50 g/L of peptone, 20 g/L of yeast extract, 4 g/L of glucose and 12 g/L of agar).

In an embodiment, in step (S1), the fermentation medium is a modified rice culture medium, a modified millet culture medium, a modified wheat culture medium, a modified corn medium, a modified sorghum culture medium or a YPD liquid medium.

In an embodiment, the modified medium is based on an original medium (a weight-volume ratio of grain to water is (0.9-1.0):(1.0-1.2)) with an addition of I-3 wt. % sea salt, 0.2-0.5 wt. % peptone and 0.1-0.2 wt. % yeast extract.

In an embodiment, in step (S1), when the seed medium is solid, the culture is performed statically at a temperature of 15-30° C. for 14-35 days; when the seed medium is liquid, the culture is performed under shaking at 100-250 rpm and 15-30° C. for 5-20 days.

In an embodiment, in step (S5), a chromatographic column is RP-C18 (250×10 mm, 5 μm); a detection wavelength is 210 nm; a mobile phase is 60% methanol/water, and a flow rate is 4 mL/min.

In an embodiment, in step (S5), fractions with a retention time of 11.9-12.3 min are collected, and further purified by HPLC to obtain compounds I-5 and I-8; the fraction with a retention time of 13.6 min is collected to obtain compound I-6; fractions with a retention time of 15.2-16.5 min are collected, and purified by HPLC to obtain compounds I-9 and I-4; and the fraction with a retention time of 18.2 min is collected to obtain compound I-14.

In an embodiment, in step (S6), the chromatographic column is RP-C18 (250×10 mm, 5 μm); a detection wavelength is 210 nm; a mobile phase is 65% methanol/water, and a flow rate is 4 mL/min.

In an embodiment, in step (S6), the fraction with a retention time of 13.4 min is collected to obtain compound I-10; the fraction with a retention time of 16.8 min is collected to obtain compound I-11; the fraction with a retention time of 20.5 min is collected to obtain compound I-1, and the fraction with a retention time of 25.6 min is collected to obtain compound I-3.

In an embodiment, in step (S7), the chromatographic column is RP-C18 (250×10 mm, 5 μm); a detection wavelength is 210 nm; a mobile phase is 50% acetonitrile/water, and a flow rate is 4 mL/min.

In an embodiment, the fraction with a retention time of 13.4 min is collected to obtain compound I-12; the fraction with a retention time of 16.7 min is collected to obtain compound I-15; the fraction with a retention time of 19.5 min is collected to obtain compound I-7, and the fraction with a retention time of 22.3 min is collected to obtain compound I-2.

In a third aspect, this application provides a method for treating inflammation in a subject in need thereof, comprising:

administering a therapeutically effective amount of the isaridin cyclodepsipeptide derivative or a pharmaceutically-acceptable salt thereof to the subject.

In a fourth aspect, this application provides a method for treating thrombosis in a subject in need thereof, comprising:

administering a therapeutically effective amount of the isaridin cyclodepsipeptide derivative or a pharmaceutically-acceptable salt thereof to the subject.

In a fifth aspect, this application provides an anti-inflammatory and/or anti-thrombotic drug containing the isaridin cyclodepsipeptide derivative or a pharmaceutically-acceptable salt thereof.

Compared to the prior art, the present disclosure has the following beneficial effects.

The isaridin cyclodepsipeptide derivatives provided herein have been experimentally proved to be capable of significantly inhibiting the nitric oxide (NO) release from lipopolysaccharide (LPS)-induced RAW264.7 cells, exhibiting good anti-inflammatory activity. Moreover, the isaridin cyclodepsipeptide derivatives can also significantly inhibit the adenosine diphosphate (ADP)-induced platelet aggregation in vitro, exhibiting good anti-thrombotic activity. Their anti-inflammatory and antithrombotic activities are better than those of positive control drugs. Additionally, the isaridin cyclodepsipeptide derivatives of the present disclosure are isolated from ascidian-associated fungi and can be prepared by microbial fermentation, simplifying the production process, shortening the production cycle and reducing the production cost. In addition, the natural compounds isolated from marine-derived fungi are not easy to produce resistance and have high safety. The compounds of the present disclosure have also been demonstrated to have low cytotoxicity, and thus have brilliant application prospects.

### BRIEF DESCRIPTION OF THE DRAWINGS

FIG. 1 is a Single-crystal X-ray diffraction pattern of compound I-2 prepared in Example 1 of the present disclosure.

FIG. 2 is a Single-crystal X-ray diffraction pattern of compound I-13 prepared in Example 1 of the present disclosure.

### DETAILED DESCRIPTION OF EMBODIMENTS

The technical solutions of the present disclosure will be described completely and clearly below with reference to the accompanying drawings and embodiments. Obviously, provided below are merely some embodiments of the disclosure, which are not intended to limit the disclosure. Unless otherwise specified, the following experiments are all performed by using conventional methods, and the equipment and reagents used in the following examples are all commercially available.

Seed medium: 10 g of yeast extract, 20 g of peptone, 20 g of glucose and 1 L of water;

Fermentation medium: 90 g of rice, 3 g of sea salt, 0.5 g of peptone, 0.2 g of yeast extract and 100 mL of water.

### Example 1 Preparation of Isaridin Cyclodepsipeptide Derivatives

The ascidian-associated fungus Beauveria felina SYSU-MS7908 has been deposited in the GDMCC (5th floor, experimental building, No. 100, Xianlie Middle Road, Guangzhou, Guangdong Province, China), and has a deposit number of GDMCC No: 61059. The Beauveria felina SYSU-MS7908 was employed to conduct fermentation, and a fermentation liquid was collected, and subjected to separation and extraction to obtain compounds I-1 to I-15.

The fermentation, separation and extraction were specifically described as follows.

1. Seed culture

1.1 A seed medium containing 10 g of yeast extract, 20 g of peptone, 20 g of glucose and 1 L of water was evenly loaded into five 500 mL conical flasks, and sterilized at 121° C. for 15 min.

1.2 The marine-derived ascidian-associated fungus Beauveria felina SYSU-MS7908 was seeded into the seed medium, and cultured at 28° C. and 180 rpm for 120 h to obtain a seed culture liquid.

2. Fermentation

2.1 Fermentation medium

90 g of rice, 3 g of sea salt, 0.5 g of peptone and 0.2 g of yeast extract were dissolved in 100 mL of water in each 1 L conical flask.

2.2 Fermentation

5 mL of the seed culture liquid was aseptically inoculated into the conical flask containing the fermentation medium and cultured at 25° C. for 28 days.

3. Isolation and purification

The Beauveria felina SYSU-MS7908 cells were subjected to extraction in methanol and concentration under reduced pressure at a temperature lower than 50° C. to obtain 105 g of an extract. The extract was separated by silica gel column chromatography gradient elution sequentially with a series of ethyl acetate-petroleum ether solutions (respectively containing 10%, 20%, 30%, 45%, 60% and 100% by volume of ethyl acetate) and 5% and 10% methanol-ethyl acetate solutions, and eight fractions were correspondingly collected (i.e., Fr. A-Fr. H).

The fractions Fr. C and Fr. D eluted respectively by 30% and 45% ethyl acetate-petroleum ether solutions were collected and subjected to reverse-phase silica gel column chromatography using gradient elution with 30%, 50%, 70% and 90% methanol/water solutions. The fractions respectively eluted with 50%, 70% and 90% methanol/water solutions were collected, dissolved in hot ethanol and recrystallized to obtain compound I-13. The remaining fractions Fr. C-L1 and Fr. D-L1 were collected.

The fraction Fr. C-L1 was allowed to pass through Sephadex LH-20 (a volume ratio of dichloromethane to methanol was 1:1), and then purified by RP-HPLC, where the chromatographic column was RP-C18 (250×10 mm, 5 μm); a detection wavelength was 210 nm; a mobile phase was 60% methanol/water, and a flow rate was 4 mL/min. Fractions with a retention time of 11.9-12.3 min were collected and further purified by HPLC to obtain compounds I-5 and I-8; the fraction with a retention time of 13.6 min was collected to obtain compound I-6; fractions with a retention time of 15.2-16.5 min were collected and further purified by HPLC to obtain compounds I-9 and I-4; and the fraction with a retention time of 18.2 min was collected to obtain compound I-14.

The fraction Fr. D-L1 was allowed to pass through Sephadex LH-20 (a volume ratio of dichloromethane to methanol was 1:1), and then purified by RP-HPLC, where the chromatographic column was RP-C18 (250×10 mm, 5 μm); a detection wavelength was 210 nm; a mobile phase was 65% methanol/water, and a flow rate was 4 mL/min. The fraction with a retention time of 13.4 min was collected to obtain compound I-10; the fraction with a retention time of 16.8 min was collected to obtain compound I-11; the fraction with a retention time of 20.5 min was collected to obtain compound I-1, and the fraction with a retention time of 25.6 min was collected to obtain compound I-3.

The fraction Fr. E eluted by 60% ethyl acetate-petroleum ether solution was allowed to pass through Sephadex LH-20 (methanol) and treated by reversed-phase silica gel column chromatography using gradient elution with 30%, 50%, 70% and 90% methanol/water solutions, and the fraction eluted by the 70% methanol/water solution was collected and subjected to RP-HPLC, where the chromatographic column was RP-C18 (250×10 mm, 5 μm); a detection wavelength was 210 nm; a mobile phase was 50% methanol/water, and a flow rate was 4 mL/min. The fraction with a retention time of 13.4 min was collected to obtain compound I-12; the fraction with a retention time of 16.7 min was collected to obtain compound I-15; the fraction with a retention time of 19.5 min was collected to obtain compound I-7, and the fraction with a retention time of 22.3 min was collected to obtain compound I-2.

Compounds I-1 to I-15 were structurally shown as follows:

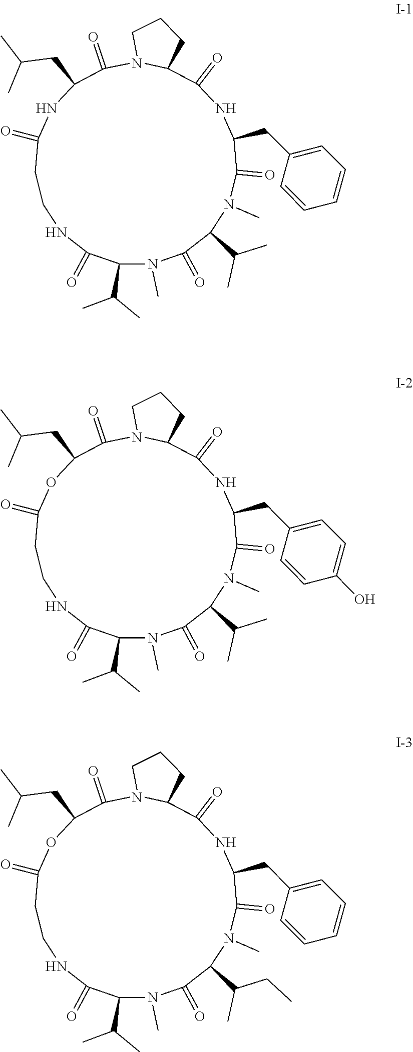 [SMILES: CC(C)C[C@@H]1NC(=O)CCNC(=O)[C@H](C(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)N(C)C(=O)[C@H](Cc2ccc(O)cc2)NC(=O)[C@@H]2CCCN2C1=O ; CCC(C)[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C]

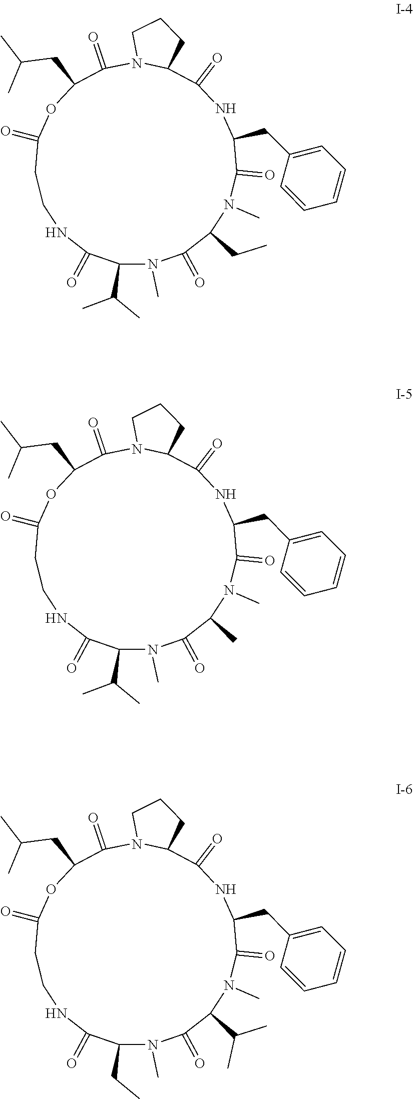 [SMILES: CC[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C ; CC(C)C[C@@H]1OC(=O)CCNC(=O)[C@H](C(C)C)N(C)C(=O)[C@H](C)N(C)C(=O)[C@H](Cc2ccccc2)NC(=O)[C@@H]2CCCN2C1=O ; CC[C@H]1C(=O)NCCC(=O)O[C@@H](CC(C)C)C(=O)N2CCC[C@H]2C(=O)N[C@@H](Cc2ccccc2)C(=O)N(C)[C@@H](C(C)C)C(=O)N1C]

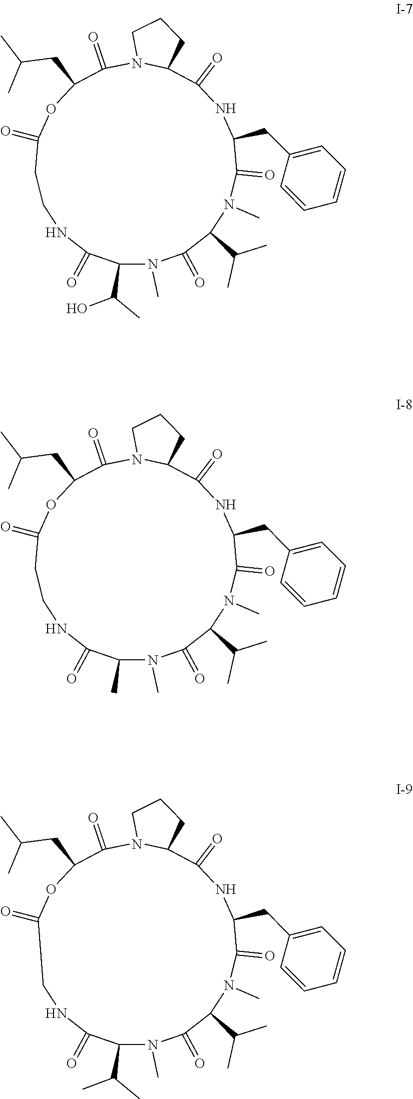 [SMILES: CC(C)C[C@@H]1OC(=O)CCNC(=O)[C@H](C(C)O)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CNC(=O)[C@H](C(C)C)N(C)C(=O)[C@H](C(C)C)N(C)C(=O)[C@H](Cc2ccccc2)NC(=O)[C@@H]2CCCN2C1=O]

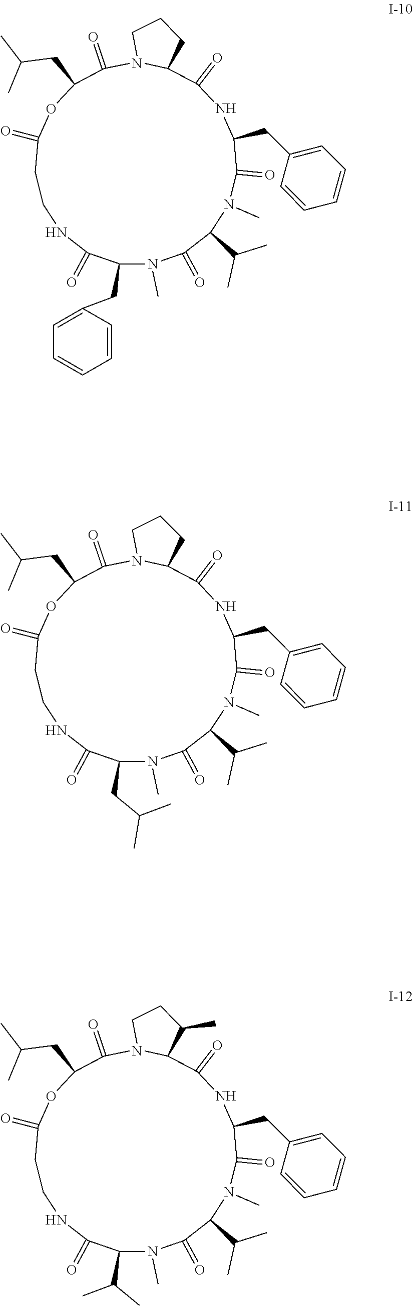 [SMILES: CC(C)C[C@@H]1OC(=O)CCNC(=O)[C@H](Cc2ccccc2)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](CC(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)N(C)C(=O)[C@H](Cc2ccccc2)NC(=O)[C@@H]2[C@H](C)CCN2C1=O]

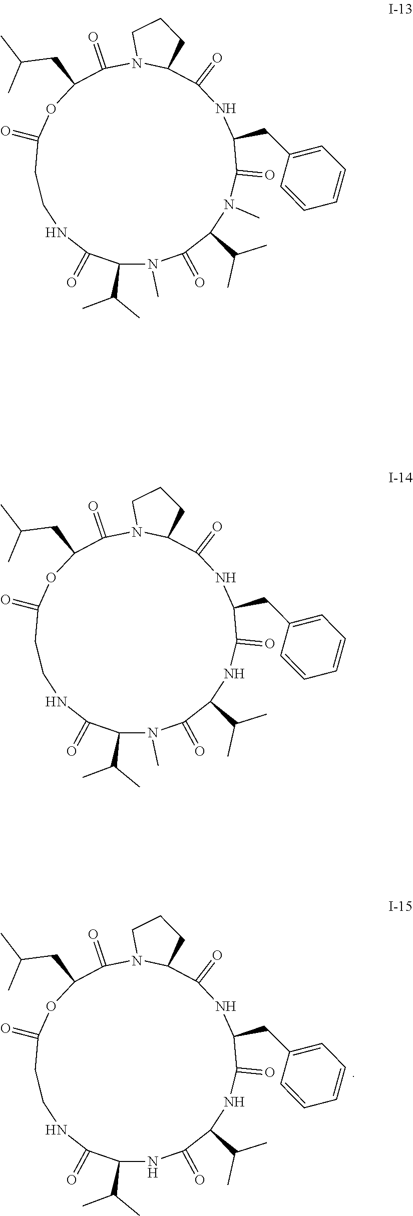 [SMILES: CC(C)C[C@@H]1OC(=O)CCNC(=O)[C@H](C(C)C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)N(C)C(=O)[C@H](C(C)C)NC(=O)[C@H](Cc2ccccc2)NC(=O)[C@@H]2CCCN2C1=O ; CC(C)C[C@@H]1OC(=O)CCNC(=O)[C@H](C(C)C)NC(=O)[C@H](C(C)C)NC(=O)[C@H](Cc2ccccc2)NC(=O)[C@@H]2CCCN2C1=O]

The isaridin derivatives were physically and chemically characterized as follows.

Compound I-1: white powder; mp 129-132° C.; [α]_{D}²⁵−164.3 (c 0.08, MeOH); UV (MeOH) λₘₐₓ (log ε) 201 (2.10) nm; IR (neat) νₘₐₓ 3278, 2956, 2877, 1620, 1543, 1446 cm⁻¹; ¹H and ¹³C NMR data was shown in Table 1; HRESIMS m/z 655.41752 [M+H]⁺ (calcd for C₃₅H₅₅O₆N₆, 655.41776).

Compound I-2: colourless crystal; mp 145-157° C.; [α]_{D}²⁵−122.2 (c 0.59, MeOH); UV (MeOH) λₘₐₓ (log ε) 201 (2.06), 266 (0.66), 277 (0.08) nm; IR (neat) νₘₐₓ 3496 (br), 3270 (br), 2950, 1722, 1680, 1645, 1608, 1516, 1238, 1167 cm⁻¹; ¹H and ¹³C NMR data was shown in Table 1; HRESIMS m/z 628.37022 [M+H]⁺ (calcd for C₃₃H₅₀O₇N₅, 628.37048).

Compound I-3: white powder; mp 186-190° C.; [α]_{D}²⁵−133.9 (c 0.64, MeOH); UV (MeOH) λₘₐₓ (log ε) 201 (2.10) nm; IR (neat) νₘₐₓ 3350, 3292, 2958, 2871, 1724, 1665, 1624, 1527, 1417, 1172 cm⁻¹; ¹H and ¹³C NMR data was shown in Table 1; HRESIMS m/z 670.41704 [M+H]⁺ (calcd for C₃₆H₅₆O₇N₅, 670.41743).

Compound I-4: white powder; mp 154-156° C.; [α]_{D}²⁵−133.9 (c 0.64, MeOH); UV (MeOH) λₘₐₓ (log ε) 201 (2.10) nm; IR (neat) νₘₐₓ 3538 (br), 3348 (br), 3296 (br), 2964, 2871, 1728, 1691, 1645, 1548, 1238, 1180 cm⁻¹; ¹H and ¹³C NMR data was shown in Table 2; HRESIMS m/z 642.38629 [M+H]⁺ (calcd for C₃₄H₅₂O₇N₅, 642.38613).

Compound I-5: white powder; mp 160-163° C.; [α]_{D}²⁵−180.4 (c 0.05, MeOH); UV (MeOH) λₘₐₓ (log ε) 201 (2.10) nm; IR (neat) νₘₐₓ 3350, 3292, 2958, 2871, 1724, 1665, 1624, 1527, 1417, 1172 cm⁻¹; ¹H and ¹³C NMR data was shown in Table 2; HRESIMS m/z 628.37055 [M+H]⁺ (calcd for C₃₃H₅₀O₈N₅, 628.37048).

Compound I-6: white powder; mp 105-107° C.; [α]_{D}²⁵−115.2 (c 0.70, MeOH); UV (MeOH) λₘₐₓ (log ε) 201 (2.06) nm; IR (neat) νₘₐₓ 3359, 3282, 2958, 2873, 1724, 1668, 1620, 1520, 1450, 1169 cm⁻¹; ¹H and ¹³C NMR data was shown in Table 2; HRESIMS m/z 642.38590 [M+H]⁺ (calcd for C₃₄H₅₂O₇N₅, 642.38613).

Compound I-7: white powder; mp 123-125° C.; [α]_{D}²⁵−139.0 (c 0.27, MeOH); UV (MeOH) λₘₐₓ (log ε) 201 (2.10) nm; IR (neat) νₘₐₓ 3351, 2962, 2873, 1724, 1666, 1521, 1448, 1342, 1170 cm⁻¹; ¹H and ¹³C NMR data was shown in Table 2; HRESIMS m/z 658.38091 [M+H]⁺ (calcd for C₃₄H₅₂O₈N₅, 658.38104).

Compound I-8: white powder; mp 154-156° C.; [α]_{D}²⁵−133.9 (c 0.64, MeOH); UV (MeOH) λₘₐₓ (log ε) 201 (2.10) nm; IR (neat) νₘₐₓ 3538 (br), 3348 (br), 3296 (br), 2964, 2871, 1728, 1691, 1645, 1548, 1238, 1180 cm⁻¹; ¹H and ¹³C NMR data was shown in Table 3; HRESIMS m/z 670.38274 [M+H]⁺ (calcd for C₃₅H₅₂O₈N₅, 670.38214).

Compound I-9: white powder; mp 135-137° C.; [α]_{D}²⁵−121.5 (c 0.32, MeOH); UV (MeOH) λₘₐₓ (log ε) 201 (2.10) nm; IR (neat) νₘₐₓ 3350, 3292, 2958, 2871, 1724, 1665, 1624, 1527, 1417, 1172 cm⁻¹; ¹H and ¹³C NMR data was shown in Table 3; HRESIMS m/z 642.38602 [M+H]⁺ (calcd for C₃₄H₅₂O₇N₅, 642.38613).

Compound I-12: colorless crystal; mp 136-139° C.; [α]_{D}²⁵−62.8 (c 0.12, MeOH); ¹H NMR (400 MHz, CDCl₃) δ 8.14 (d, J=7.5 Hz, 1H), 7.42 (d, J=10.2 Hz, 1H), 7.27 (m, 2H), 7.25 (m, 2H), 7.25 (m, 1H), 5.34 (d, J=10.2 Hz, 1H), 5.12 (d, J=10.7 Hz, 1H), 4.65 (ddd, J=10.9, 7.5, 5.0 Hz, 1H), 4.29 (d, J=10.7 Hz, 1H), 4.15 (m, 1H), 3.71 (d, J=2.4 Hz, 1H), 3.66 (m, 1H), 3.26 (m, 1H), 3.17 (m, 1H), 3.14 (s, 3H), 3.01 (m, 1H), 2.97 (s, 3H), 2.63 (dd, J=11.6, 2.8 Hz, 1H), 2.48 (m, 1H), 2.44 (m, 1H), 2.39 (m, 1H), 2.48 (m, 1H), 1.96 (m, 1H), 1.96 (m, 1H), 1.56 (m, 1H), 1.44 (m, 1H), 1.24 (m, 1H), 1.06 (d, J=7.0 Hz, 3H), 1.01 (d, J=3.2 Hz, 3H), 0.99 (d, J=3.2 Hz, 3H), 0.92 (d, J=6.4 Hz, 3H), 0.89 (d, 3H), 0.87 (d, 3H), 0.87 (d, 3H); ¹³C NMR (101 MHz, CDCl₃) δ 19.0, 19.3, 19.6, 19.8, 20.4, 20.6, 23.5, 24.9, 27.8, 27.8, 29.2, 29.8, 30.2, 35.2, 35.5, 35.7, 38.9, 40.1, 45.6, 53.9, 57.7, 68.1, 66.6, 73.5, 127.4, 128.8, 128.9, 136.5, 168.8, 169.9, 170.1, 172.2, 173.8, 174.2. HRESIMS m/z 670.42432 [M+H]⁺ (calcd for C₃₆H₅₆O₇N₅, 670.42258).

Compound I-13: colorless crystal; mp 198-200° C.; [α]_{D}²⁵−143.8 (c 0.18, MeOH); ¹H NMR (400 MHz, CDCl₃) δ 8.14 (d, J=7.5 Hz, 1H), 7.42 (d, J=10.2 Hz, 1H), 7.27 (m, 2H), 7.25 (m, 2H), 7.25 (m, 1H), 5.34 (d, J=10.2 Hz, 1H), 5.12 (d, J=10.7 Hz, 1H), 4.65 (ddd, J=10.9, 7.5, 5.0 Hz, 1H), 4.29 (d, J=10.7 Hz, 1H), 4.15 (m, 1H), 4.09 (d, J=7.6 Hz, 1H), 3.50 (dd, J=9.8, 6.4 Hz, 2H), 3.17 (m, 1H), 3.14 (s, 3H), 3.01 (m, 1H), 2.97 (s, 3H), 2.63 (dd, J=11.6, 2.8 Hz, 1H), 2.48 (m, 1H), 2.44 (m, 1H), 2.39 (m, 1H), 2.22 (mp, 1H), 2.13 (m, 1H), 1.96 (m, 1H), 1.96 (m, 1H), 1.77 (m, 1H), 1.30 (m, 1H), 1.24 (m, 1H), 1.01 (d, J=3.2 Hz, 3H), 0.99 (d, J=3.2 Hz, 3H), 0.92 (d, J=6.4 Hz, 3H), 0.89 (d, 3H), 0.87 (d, 3H), 0.87 (d, 3H); ¹³C NMR (101 MHz, CDCl₃) δ 19.0, 19.6, 19.8, 20.4, 20.6, 22.1, 23.5, 24.9, 27.8, 27.8, 29.2, 29.8, 32.4, 35.2, 35.5, 35.7, 38.9, 47.3, 53.9, 57.7, 61.1, 66.6, 73.5, 127.4, 128.8, 128.9, 136.5, 168.8, 169.9, 170.1, 172.2, 173.8, 174.2. HRESIMS m/z 656.40183 [M+H]⁺ (calcd for C₃₅H₅₄O₇N₅, 656.40178).

Compound I-14: white powder; mp 138-140° C.; [α]_{D}²⁵−103.6 (c 0.85, MeOH); ¹H NMR (CDCl₃, 400 MHz) δ_{H}: 8.07 (1H, d, J 8.1), 7.23 (2H, m), 7.17 (3H, m), 6.98 (1H, d, J 8.7), 5.19 (1H, d, J 9.5), 4.71 (1H, m), 4.48 (1H, t, J 9.2), 4.33 (1H, d, J 10.7), 4.11 (1H, d, J 8.3), 3.50-3.45 (1H, m), 3.44 (1H, m), 3.21 (1H, t, J 12.5), 3.09 (1H, dd, J 14.4, 5.9), 2.99-2.93 (1H, m), 2.92 (3H, s), 2.64 (1H, d, J 15.7), 2.53 (1H, dd, J 12.1, 3.5), 2.49 (1H, m), 2.24 (1H, m), 2.16-2.10 (1H, m), 2.09 (1H, m), 2.00 (1H, m), 1.93 (2H, m), 1.73 (1H, m), 1.30-1.24 (1H, m), 0.98 (3H, d, J 6.5), 0.96-0.91 (9H, m), 0.88 (6H, t, J 6.4); ¹³C NMR (101 MHz, CDCl₃) δ 18.9, 19.6, 19.7, 20.3, 21.0, 21.9, 23.4, 25.0, 27.1, 29.3, 31.6, 32.2, 35.0, 35.3, 37.4, 39.0, 47.2, 55.0, 55.2, 61.1, 66.6, 73.3, 127.2, 128.8, 129.0, 136.7, 168.5, 169.9, 171.6, 172.1, 172.8, 173.7. HRESIMS m/z 642.38624 [M+H]⁺ (calcd for C₃₄H₅₂O₇N₅, 642.38613).

Compound I-15: white powder; mp 131-133° C.; [α]_{D}²⁵−64.2 (c 0.11, MeOH); UV (MeOH) λₘₐₓ (log ε) 201 (2.10) nm; IR (neat) νₘₐₓ 3282, 2956, 1728, 1639, 1541, 1444 cm⁻¹; ¹H and ¹³C NMR data was shown in Table 1; HRESIMS m/z 628.37012 [M+H]⁺ (calcd for C₃₃H₅₀O₇N₅, 628.37048).

The NMR data of compounds I-1 to I-9 and I-15 were shown in Tables I-3.

TABLE 1
NMR data of compounds I-1 to I-3 and I-15 (100 MHz/400 MHz, CDCl₃/DMSO-d6, ppm)

| | I-15 | | | I-1 | | | I-2 | | | I-3 | | |
|---|---|---|---|---|---|---|---|---|---|---|---|---|
| NO. | δ_{C}, | δ_{H}, | | δ_{C}, | δ_{H}, | | δ_{C}, | δ_{H}, | | δ_{C}, | δ_{H}, | |
| | type | mult, J (Hz) | | type | mult, J (Hz) | | type | mult, J (Hz) | | type | mult, J (Hz) | |
| | HMPA¹ | | | Leu¹ | | | HMPA¹ | | | HMPA¹ | | |
| CO | 169.8, C | | | 171.3, C | | | 170.4, C | | | 169.0, C | | |
| α | 72.6, CH | 5.04, | d (11.0) | 52.2, CH | 4.66, | dd (11.3; | 73.4, CH | 5.29, | dd (11.2, | 73.0, CH | 5.26, | d (10.7) |
| | | | | | | 1.8) | | | 1.8) | | | |
| β | 39.6, CH₂ | 1.95, | m | 38.8, CH₂ | 1.76, | m | 38.7, CH₂ | 1.93, | m | 37.5, CH₂ | 1.68, | m |
| | | 1.21, | m | | 1.34, | m | | 1.25, | m | | 1.53, | m |
| γ | 24.7, CH | 1.95, | m | 25.4, CH | 1.74, | m | 24.8, CH | 1.93, | m | 24.3, CH | 1.85, | m |
| δ | 23.5, CH₃ | 0.98, | d (3.3) | 23.7, CH₃ | 1.00, | d (3.3) | 23.4, CH₃ | 0.97, | d (3.3) | 23.1, CH₃ | 0.94, | d (3.8) |
| δ′ | 21.4, CH₃ | 0.93, | d (3.3) | 20.5, CH₃ | 1.00, | d (3.3) | 20.5, CH₃ | 0.97, | d (3.3) | 20.0, CH₃ | 0.94, | d (3.8)) |
| NH | | | | | 6.29, | s | | | | | | |
| | Pro² | | | Pro² | | | Pro² | | | Pro² | | |
| CO | 172.0, C | | | 172.6, C | | | 171.8, C | | | 171.4, C | | |
| α | 60.9, CH | 4.11, | d(8.3) | 61.2, CH | 4.16, | dd (8.6; | 61.1, CH | 4.09, | m | 60.2, CH | 4.14, | d (8.3) |
| | | | | | | 2.0) | | | | | | |
| β | 32.0, CH₂ | 2.33, | m | 32.5, CH₂ | 2.24, | m | 32.3, CH₂ | 2.22, | m | 31.7, CH₂ | 2.13, | m |
| | | 2.09, | m | | 2.10, | m | | 2.11, | m | | 1.98, | m |
| γ | 21.5, CH₂ | 1.74, | m | 22.2, CH₂ | 1.74, | m | 22.0, CH₂ | 1.76, | m | 21.5, CH₂ | 1.70, | m |
| | | 1.31, | m | | 1.34, | m | | 1.36, | m | | 1.10, | m |
| δ | 47.1, CH₂ | 3.42, | m | 47.4, CH₂ | 3.52, | m | 47.3, CH₂ | 3.49, | m | 46.8, CH₂ | 3.30, | m |
| | Phe³ | | | Phe³ | | | Tyr³ | | | Phe³ | | |
| CO | 170.7, C | | | 174, C | | | 173.8, C | | | 173.5, C | | |
| α | 54.9, CH | 5.11, | m | 54.2, CH | 4.66, | m | 54.1, CH | 4.57, | m | 53.1, CH | 4.62, | m |
| β | 38.3, CH₂ | 3.16, | m | 35.1, CH₂ | 2.95, | m | 34.4, CH₂ | 2.93, | m | 34.1, CH₂ | 2.82, | m; |
| | | | | | | | | | | | 3.08, | m |
| Φ1′ | 136.5, C | | | 137.1, C | | | 127.2, C | | | 136.9, C | | |
| Φ3′, | 129.2, CH | 7.12, | m | 129.1, CH | 7.27, | m | 115.7, CH | 6.78, | d (8.2) | 129.0, CH | 7.29, | m |
| Φ5′ | | | | | | | | | | | | |
| Φ2′, | 128.7, CH | 7.08, | m | 128.7, CH | 7.27, | m | 129.8, CH | 7.06, | d (8.2) | 128.3, CH | 7.29, | m |
| Φ6′ | | | | | | | | | | | | |
| Φ4′ | 127.3, CH | 7.24, | m | 127.2, CH | 7.22, | m | 156.2, C | | | 126.8, CH | 7.25, | m |
| NH | | 8.32, | d (8.4) | | 8.96, | d (7.5) | | 8.07, | d (7.5) | | 7.92, | d (7.6) |
| | Val⁴ | | | NMe-Val⁴ | | | NMe-Val⁴ | | | NMe-Ile⁴ | | |
| CO | 173.9, C | | | 170.0, C | | | 170.0, C | | | 168.9, C | | |
| α | 58, CH | 4.42, | dd (9.1, | 57.7, CH | 5.13, | d (10.7) | 57.6, CH | 5.08, | d (10.6) | 54.3, CH | 5.16, | d (10.9) |
| | | | 3.2) | | | | | | | | | |
| β | 28.8, CH | 2.66, | m | 27.7, CH | 2.41, | m | 27.7, CH | 2.36, | m | 32.4, CH | 2.20, | m |
| γ | 16.9, CH₃ | 0.81, | d (6.8) | 18.9, CH₃ | 0.86, | d (6.8) | 18.9, CH₃ | 0.84, | d (6.6) | 23.7, CH₂ | 1.40, | m |
| γ′ | 20.0, CH₃ | 0.93, | d (6.8) | 20.4, CH₃ | 1.00, | d (6.8) | 20.3, CH₃ | 0.84, | d (6.8) | 15.7, CH₃ | 0.77, | d (6.8) |
| δ | | | | | | | | | | 9.3, CH₃ | 0.75, | d (6.8) |
| NH/NMe | | 6.09, | d (9.1) | 29.7, CH₃ | 3.12, | s | 29.7, CH₃ | 3.06, | s | 29.4, CH₃ | 3.00, | s |
| | Val⁵ | | | NMe-Val⁵ | | | NMe-Val⁵ | | | NMe-Val⁵ | | |
| CO | 170.7, C | | | 168.9, C | | | 168.8 C | | | 167.9, C | | |
| α | 62.0, CH | 3.90, | d (4.0) | 66.4, CH | 4.35, | d (10.7) | 66.5, CH | 4.27, | d (10.7) | 65.6, CH | 4.31, | d (10.7) |
| β | 29.0 CH | 2.31, | m | 27.7, CH | 2.44, | m | 27.7, CH | 2.41, | m | 27.2, CH | 2.31, | m |
| γ | 17.6, CH₃ | 0.95, | d (6.8) | 19.7, CH₃ | 0.86, | d (6.8) | 19.5, CH₃ | 0.88, | d (6.6) | 19.1, CH₃ | 0.86, | d (6.8) |
| γ′ | 19.7, CH₃ | 0.93, | d (6.8) | 19.8, CH₃ | 0.86, | d (6.8) | 19.5, CH₃ | 0.84, | d (6.6) | 19.4, CH₃ | 0.82, | d (6.8) |
| NH/NMe | | 7.71, | s | 29.2, CH₃ | 2.97, | s | 29.2, CH₃ | 2.93, | s | 28.7, CH₃ | 2.82, | s |
| | β-Ala⁶ | | | β-Ala⁶ | | | β-Ala⁶ | | | β-Ala⁶ | | |
| CO | 173.4, C | | | 172.2, C | | | 174.2, C | | | 173.7, C | | |
| α | 35.9, CH₂ | 4.04, | m | 35.4, CH₂ | 4.11, | m | 35.6, CH₂ | 4.08, | m | 35.4, CH₂ | 3.97, | m |
| | | 3.35, | m | | 3.16, | m | | 3.07, | m | | 3.08 | m |
| β | 35.9, CH₂ | 2.73, | dd (14.2; | 36.9, CH₂ | 2.48, | dt (14.5; | 35.2, CH₂ | 2.51, | dt (14.5; | 34.9, CH₂ | 2.72, | dt (14.5; |
| | | | 7.2) | | | 3.1) | | | 3.1) | | | 3.1) |
| | | 2.40, | m | | 2.14, | m | | 2.41, | m | | 2.33, | m |
| NH | | 6.87, | m | | 7.38, | d (10.1) | | 7.39, | d (10.1) | | 7.31, | d (10.1) |

TABLE 2
NMR data of compounds I-4 to I-7 (100 MHz/400 MHz, CDCl₃, ppm)

| NO. | I-4 | | | I-5 | | | I-6 | | | I-7 | | |
|---|---|---|---|---|---|---|---|---|---|---|---|---|
| | δ_{C}, | δ_{H}, | | δ_{C}, | δ_{H}, | | δ_{C}, | δ_{H}, | | δ_{C}, | δ_{H}, | |
| | type | mult, J (Hz) | | type | mult, J (Hz) | | type | mult, J (Hz) | | type | mult, J (Hz) | |
| | HMPA¹ | | | HMPA¹ | | | HMPA¹ | | | HMPA¹ | | |
| CO | 170.0, C | | | 169.9, C | | | 169.9, C | | | 170.0, C | | |
| α | 73.2, CH | 5.20, | m | 73.1, CH | 5.06, | m | 72.9, CH | 5.20, | m | 73.3, CH | 5.27, | m |
| β | 38.8, CH₂ | 1.93, | m | 38.8, CH₂ | 1.93, | m | 38.5, CH₂ | 1.95, | m | 38.7, CH₂ | 1.96, | m |
| | | 1.24, | m | | 1.26, | m | | 1.26, | m | | 1.28, | m |
| γ | 24.9, CH | 1.95, | m | 25.0, CH | 1.91, | m | 24.7, CH | 1.94, | m | 24.9, CH | 1.96, | m |
| δ | 23.5, CH₃ | 0.98, | d (3.5) | 23.4, CH₃ | 0.96, | d (3.3) | 23.2, CH₃ | 0.98, | d (3.3) | 23.4, CH₃ | 1.00, | d (3.3) |
| δ′ | 20.8, CH₃ | 0.96, | d (3.5) | 21.2, CH₃ | 0.92, | d (3.3) | 20.5, CH₃ | 0.96, | d (3.3) | 20.6, CH₃ | 1.00, | d (3.3) |
| | Pro² | | | Pro² | | | Pro² | | | Pro² | | |
| CO | 171.9, C | | | 171.2, C | | | 171.7, C | | | 171.9, C | | |
| α | 61.0, CH | 4.09, | d (8.2) | 61.0, CH | 4.07, | m | 60.8, CH | 4.10, | dd (8.6; 2.0) | 60.9, CH | 4.10, | m |
| β | 32.3, CH₂ | 2.23, | m | 32.1, CH₂ | 2.25, | m | 32.1, CH₂ | 2.24, | m | 32.3, CH₂ | 2.23, | m |
| | | 2.11, | m | | 2.01, | m | | 2.12, | m | | 2.13, | m |
| γ | 22.3, CH₂ | 1.74, | m | 21.8, CH₂ | 1.71, | m | 21.8, CH₂ | 1.77, | m | 22.0, CH₂ | 1.78, | m |
| | | 1.26, | m | | 1.11, | m | | 1.29, | m | | 1.31, | m |
| δ | 47.3, CH₂ | 3.48, | m | 47.2,CH₂ | 3.43, | m | 47.1, CH₂ | 3.50, | m | 47.2, CH₂ | 3.50, | m |
| | Phe³ | | | Phe³ | | | Phe³ | | | Phe³ | | |
| CO | 173.8, C | | | 173.1, C | | | 173.8, C | | | 170.9, C | | |
| α | 53.5, CH | 4.75, | m | 53.1, CH | 4.82, | m | 53.2, CH | 4.73, | m | 53.6, CH | 4.70, | m |
| β | 35.9, CH₂ | 3.04, | m | 36.1, CH₂ | 3.02, | dd(14.0, | 35.6, CH₂ | 2.93, | m | 35.2, CH₂ | 2.97, | m |
| | | | | | | 4.1) | | | | | | |
| | | 2.92, | m | | 2.59, | m | | 3.02, | m | | 3.03, | m |
| Φ1′ | 136.4, C | | | 136.5, C | | | 136.1, C | | | 136.3, C | | |
| Φ3′ | 129.0, CH | 7.28, | m | 129.0, CH | 7.20, | m | 128.7, CH | 7.28, | m | 128.8, CH | 7.29, | m |
| Φ5′ | | | | | | | | | | | | |
| Φ2′ | 128.9, CH | 7.20, | m | 128.9, CH | 7.29, | m | 128.7, CH | 7.20, | m | 128.8, CH | 7.29, | m |
| Φ6′ | | | | | | | | | | | | |
| Φ4′ | 127.4, CH | 7.26, | m | 127.3, CH | 7.25, | m | 127.2, CH | 7.26, | m | 127.3, CH | 7.25, | m |
| NH | | 8.09, | d (7.6) | | 8.16, | d (7.9) | | 8.08, | d (7.5) | | 8.12, | d (7.4) |
| | NMe-Abu⁴ | | | NMe-Ala⁴ | | | NMe-Val⁴ | | | NMe-Val⁴ | | |
| CO | 171.1, C | | | 172.4, C | | | 169.9, C | | | 173.8, C | | |
| α | 52.9, CH | 5.36, | d (10.7) | 47.1, CH | 5.51, | d (6.9) | 57.6, CH | 5.10, | d (10.7) | 57.8, CH | 5.09, | d (10.7) |
| β | 23.2, CH₂ | 2.05, 1.58, | m | 16.1, CH₃ | 1.43, | d (6.9) | 27.2, CH | 2.34, | m | 27.5, CH | 2.38, | m |
| γ | 10.5, CH₃ | 0.87, | d (6.8) | | | | 19.0, CH₃ | 0.90, | d (6.8) | 19.0, CH₃ | 0.88, | d (6.5) |
| γ′ | | | | | | | 19.6, CH₃ | 0.88, | d (6.8) | 20.2, CH₃ | 0.91, | d (6.5) |
| NMe | 29.6, CH₃ | 3.14, | s | 30.4, CH3 | 3.28, | s | 29.9, CH3 | 3.15, | s | 29.8, CH3 | 3.10, | s |
| | NMe-Val⁵ | | | NMe-Val⁵ | | | NMe-Abu⁵ | | | NMe-Thr⁵ | | |
| CO | 168.5, C | | | 168.5, C | | | 168.2, C | | | 168.2, C | | |
| α | 67.1, CH | 4.37, | d (10.9) | 66.9, CH | 4.44, | d (10.9) | 61.8, CH | 4.73, | d (10.7) | 66.1, CH | 4.70, | d (10.7) |
| β | 26.6, CH | 2.41, | m | 26.3, CH | 2.44, | m | 23.2, CH₂ | 2.26, 1.39 | | 64.1, CH | 4.32, | m |
| γ | 22.1, CH₃ | 0.94, | d (6.8) | 19.8, CH₃ | 0.94, | d (6.8) | 10.7, CH₃ | 0.92, | d (6.8) | 19.6, CH₃ | 1.23, | d (5.9) |
| γ′ | 19.6, CH₃ | 0.81, | d (6.8) | 19.1, CH₃ | 0.81, | d (6.8) | | | | | | |
| NMe | 28.8, CH₃ | 2.83, | s | 28.9, CH₃ | 2.78, | s | 28.6, CH₃ | 2.86, | s | 29.3, CH₃ | 3.00, | s |
| | β-Ala⁶ | | | β-Ala⁶ | | | β-Ala⁶ | | | β-Ala⁶ | | |
| CO | 174.1, C | | | 173.6, C | | | 173.5, C | | | 173.9, C | | |
| α | 35.5, CH₂ | 4.15, | m | 35.3, CH₂ | 4.09, | m | 35.5, CH₂ | 4.13, | m | 35.5, CH₂ | 4.15, | m |
| | | 3.16, | m | | 3.19, | m | | 3.19, | m | | 3.18, | m |
| β | 35.4, CH₂ | 2.59, | m | 35.3, CH₂ | 2.59, | m | 35.2, CH₂ | 2.60, | m | 35.4, CH₂ | 2.63, | d (14.7) |
| | | 2.59, | m | | 2.59, | m | | 2.60, | m | | 2.55, | m |
| NH | | 7.57, | d (7.8) | | 7.56, | d (7.9) | | 7.52, | d (10.1) | | 7.57, | d (10.0) |

TABLE 3
NMR data of compounds I-8 to I-9
(100 MHz/400 MHz, CDCl₃, ppm)

| NO. | I-8 | | | I-9 | | |
|---|---|---|---|---|---|---|
| | δ_{C}, | δ_{H}, | | δ_{C} | δ_{H} | |
| | type | mult, J (Hz) | | type | mult, J (Hz) | |
| | HMPA¹ | | | HMPA¹ | | |
| CO | 169.8, C | | | 171.3, C | | |
| α | 72.7, CH | 5.07, | m | 73.9, CH | 5.29, | m |
| β | 38.6, CH₂ | 1.93, | m | 39.6, CH₂ | 1.94, | m |
| | | 1.27, | m | | 1.40, | m |
| γ | 24.6, CH | 1.92, | m | 24.8, CH | 1.94, | m |
| δ | 23.2, CH₃ | 0.98, | d (6.5) | 23.4, CH₃ | 1.00, | d (6.5) |
| δ′ | 20.5, CH₃ | 0.98, | d (6.5) | 21.2, CH₃ | 0.96, | d (6.5) |
| | Pro² | | | Pro² | | |
| CO | 171.5, C | | | 169.6, C | | |
| α | 60.7, CH | 4.11, | d (8.2) | 61.4, CH | 4.11, | d (8.2) |
| β | 31.9, CH₂ | 2.27, | m | 32.1, CH₂ | 2.11, | m |
| | | 2.07, | m | | | |
| γ | 21.7, CH₂ | 1.74, | m | 22.1, CH₂ | 1.73, | m |
| | | 1.20, | m | | 1.20, | m |
| δ | 47.0, CH₂ | 3.48, | m | 47.3, CH₂ | 3.50, | m |
| | Phe³ | | | Phe³ | | |
| CO | 173.8, C | | | 172.3, C | | |
| α | 52.9, CH | 4.83, | ddt (10.9, | 53.6, CH | 4.74, | m |
| | | | 7.8, 4.7) | | | |
| β | 35.9, CH₂ | 2.90, | m | 35.3, CH₂ | 2.82, | m |
| | | 3.05, | dd(13.9, | | 3.03, | m |
| | | | 4.7) | | | |
| Φ1′ | 136.1, C | | | 136.6, C | | |
| Φ3′, | 128.8, CH | 7.29, | m | 128.9, CH | 7.27, | m |
| Φ5′ | | | | | | |
| Φ2′, | 128.6, CH | 7.21, | m | 128.9, CH | 7.23, | m |
| Φ6′ | | | | | | |
| Φ4′ | 127.1, CH | 7.26, | m | 127.3, CH | 7.23, | m |
| NH | | 8.12, | d (7.9) | | 7.43, | d (7.9) |
| | NMe-Val⁴ | | | NMe-Val⁴ | | |
| CO | 170.3, C | | | 170.8, C | | |
| α | 57.7, CH | 5.05, | m | 57.1, CH | 5.25, | m |
| β | 27.1, CH | 2.34, | m | 28.8, CH | 2.42, | m |
| γ | 19.1, CH₃ | 0.92, | d (6.8) | 20.2, CH₃ | 0.94, | d (6.8) |
| γ′ | 19.6, CH₃ | 0.92, | d (6.8) | 20.2, CH₃ | 0.96, | d (6.8) |
| NMe | 30.1, CH₃ | 3.21, | s | 30.3, CH₃ | 3.23, | s |
| | NMe-Ala⁵ | | | NMe-Val⁵ | | |
| CO | 169.3, C | | | 169.7, C | | |
| α | 55.5, CH | 5.06, | m | 67.1, CH | 4.03, | d (10.9) |
| β | 15.0, CH₃ | 1.37, | d(6.7) | 27.4, CH | 2.42, | m |
| γ | — | | | 20.3, CH₃ | 1.03, | d (6.8) |
| γ′ | — | | | 18.7, CH₃ | 0.89, | d (6.8) |
| NMe | 28.1, CH₃ | 2.85, | s | 30.3, CH₃ | 3.03, | s |
| | β-Ala⁶ | | | Gly⁶ | | |
| CO | 173.3, C | | | 169.5, C | | |
| α | 35.4, CH₂ | 4.07, | m | 43.9, CH₂ | 4.09, | m |
| | | 3.23, | m | | 3.85, | m |
| β | 34.8, CH₂ | 2.63, | m | — | | |
| | | 2.67, | m | — | | |
| NH | | 7.42, | dd (9.7, | | 6.95, | dd (9.6, |
| | | | 2.7) | | | 2.7) |

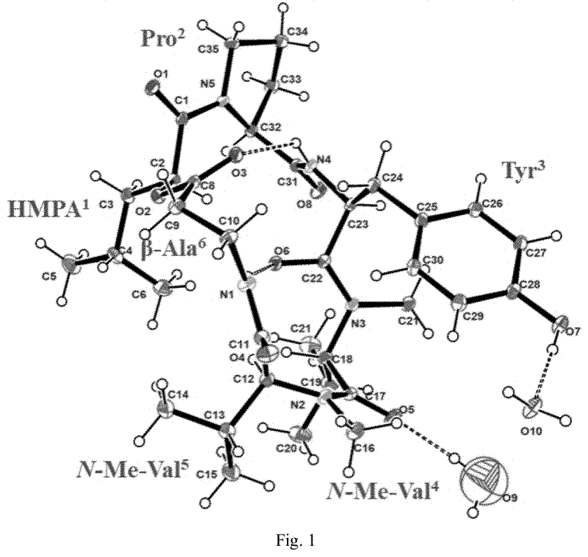

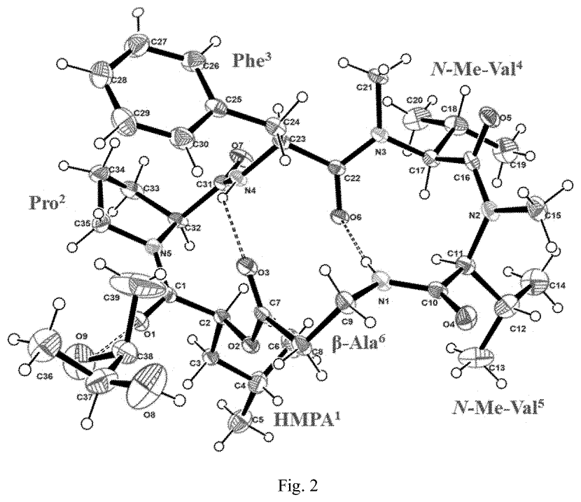

Among them, the Crystallographic Data of I-2 and I-13 was shown as follows, and their X-ray crystallographic structures were respectively shown in FIGS. 1-2.

Crystallographic Data of I-2. C₃₅H₅₃N₅O₈, Mr=671.84, monoclinic, space group P2₁, a=10.13190 (10) Å, b=36.2803 (2) Å, c=10.23540 (10) Å; a=90°, β=95.7220 (10°), γ=90°, V=3743.67 (6) Å³, T=100.0 (8) K, Z=4, ρ_{calcd}=1.256 g/m³, crystal size 0.44×0.35×0.06 mm³, F(000)=1528, 2θ range for data collection 4.872 to 149.028, absorption coefficient 0.757 mm⁻¹, reflections collected 70852, independent reflections 14639 [Rᵢₙₜ=0.0308, R_{sigma}=0.0249], final R indices [I>2σ(I)] R₁=0.0387, wR₂=0.1046. The goodness of fit on F² was 1.047. Flack parameter −0.02 (4); Hooft parameter −0.00 (3). Crystallographic data for the structure of isaridin J (2) have been deposited in the Cambridge Crystallographic Data Centre (deposition numbers: CCDC 2108990).

Crystallographic Data of I-13. C₃₅H₅₃N₅O₇, Mr=655.84, Orthorhombic, space group P2₁2₁2₁, a=17.88018 (14) Å, b=22.1738 (2) Å, c=22.7060 (2) Å; a=90°, β=90°, γ=90°, V=9002.29 (13) Å³, T=100.0 (8) K, Z=8, ρ_{calcd}=1.034 g/m³, crystal size 0.25×0.15×0.05 mm³, F(000)=3033, 20 range for data collection 7.45 to 148.174, absorption coefficient 0.592 mm⁻¹, reflections collected 36641, independent reflections 17785 [Rᵢₙₜ=0.0296, R_{sigma}=0.0385], final R indices [I>2σ(I)] R₁=0.0550, wR₂=0.1550; The goodness of fit on F² was 1.018. Flack parameter 0.02 (6); Hooft parameter −0.04 (6). CCDC deposition numbers: CCDC 2109162.

### Example 2 Anti-Inflammatory Activity Test

The effect of the compounds I-1 to I-9 and I-13 to I-15 prepared in Example 1 on the NO release from LPS-induced macrophage RAW264.7 cells was investigated to evaluate the anti-inflammatory activity, which was specifically described as follows.

1.1 Experimental Materials

Lipopolysaccharide (LPS); indomethacin (Indomethacin, Indo, positive control); mouse mononuclear macrophages (RAW264.7); dimethyl sulfoxide (DMSO); Methylthiazolyldiphenyl-tetrazolium bromide (MTT, 5 mg/mL); and Griess reagent nitrite measurement kit (Beyotime Biotechnology Co., Ltd).

1.2 Experimental Method

The compounds were respectively dissolved in DMSO to obtain a series of 10 mM stock solutions, which were diluted with Dulbecco's modified eagle medium (DMEM) to a required concentration (DMSO<2%) for use.

RAW264.7 cells (1×10⁵ cells/mL) were inoculated into a 96-well plate at 100 μL per well and incubated at 37° C. and 5% CO₂ for 12 h. Different concentrations of samples containing LPS (with a final concentration of 1 μg/mL) were respectively loaded on the plate. The experimental groups were established as follows: blank group (100 μL DMEM), LPS-induced model group (1 μL LPS+99 μL DMEM), LPS+Indo group (1 μL LPS+25 μL Indo+74 μL DMEM) and LPS+ sample group (1 μL LPS+99 μL sample-containing medium), where a concentration of lipopolysaccharide was 100 μg/mL, and a concentration of indomethacin was 200 μg/mL. After that, the 96-well plate was cultured for 24 h. 50 μL of the supernatant was carefully pipetted to another 96-well plate, added with NO I and NO II reagents from the Griess kit, mixed evenly and subjected to standing at room temperature for 10 min. After that, the absorbance of each well of the 96-well plate at 540 nm was measured with a microplate reader and then plugged into the standard curve to calculate the NO release level.

50 μL of residual culture medium was carefully pipetted, added with 100 μL of MTT solution diluted with DMEM, and cultured in an incubator for 4 h. The supernatant was pipetted, added with 110 μL of DMSO solution, shaken for 10 min and measured with a microplate reader for the absorbance at 490 nm to evaluate the cell viability.

NO release inhibition rate (%)=(OD_{LPS-induced model group}−OD_{LPS+sample group})/(OD_{LPS-induced model group}−OD blank group)×100%; and

cell viability (%)=[(mean OD of sample groups)/mean OD of control groups]×100%.

2. Results

The tested compounds all had a half maximal inhibitory concentration (IC₅₀) of 6-30 μM, which was lower than that of the positive control indomethacin (IC₅₀: 38 μM), indicating an excellent anti-inflammatory activity. In the MTT test, all compounds were confirmed to be non-cytotoxic to RAW264.7 cells, and thus have high safety.

### Example 3 In-Vitro Anti-Thrombotic Activity Test

Compounds I-1 to I-9 and I-13 to I-15 prepared in Example 1 were tested for their inhibitory effects on the ADP-induced platelet aggregation in vitro to evaluate the anti-thrombotic activities, and the test process was specifically described as follows.

Kunming mice were anesthetized with pentobarbital sodium, and blood was collected from the celiac artery, where the syringe was added with 3.2% sodium citrate for anti-coagulation in advance (a volume ratio of whole blood to the anticoagulant was 9:1). The mixture was gently mixed and centrifuged at 1000 r/min for 10 min to collect the supernatant as the platelet-rich plasma (PRP). The remaining part was centrifuged at 3000 r/min for 10 min to obtain platelet-poor plasma (PPP). A platelet count of the PRP was adjusted to 3×10⁸/mL with PPP.

For the blank control group, 1% DMSO was mixed with 295 μL of PRP; for the drug group, 5 μL of the isaridin derivatives with different concentrations (a final concentration of 0-100 μM) was mixed with 295 μL of PRP; and regarding the positive control group, aspirin was mixed with plasma to a final concentration of 120 μM. After incubated at 37° C. for 5 min, the individual mixed systems were respectively transferred to a detection hole of the platelet aggregation analyzer, and the measurement channels were zeroed with PPP in turn. After that, the sample group and the control group were respectively added with 15 μL (0.5 mg/mL) of the inducer ADP. Three parallel samples were tested for each group, and the platelet aggregation rate was measured at 37° C. by turbidimetric method, where the maximum aggregation rate within 5 min was recorded.

The platelet aggregation rate was expressed as the maximum platelet aggregation rate, and the results were expressed as the inhibition rate.

Inhibition rate (%)=(platelet aggregation rate of the blank control group−platelet aggregation rate of the drug group)/the platelet aggregation rate of the blank control group×100%.

The results showed that with the increase in the concentration of the isaridin drug, the maximum platelet aggregation rate decreases gradually, that was, in a concentration-dependent manner. The IC₅₀ values of the 12 tested isaridin compounds were approximately 10-100 μM, which were all lower than that of the positive control aspirin (IC₅₀: 120 μM), indicating that the isaridin compounds of the disclosure were superior to the aspirin in terms of the anti-thrombotic activity.

The formation of thrombus mainly includes three stages: (1) platelet adhesion and aggregation; (2) blood coagulation; and (3) fibrinolysis. Isaridin cyclodepsipeptide derivatives can inhibit the ADP-induced platelet aggregation, and further reduce the blood viscosity, directly affecting the first stage of the thrombosis. Therefore, the isaridin cyclodepsipeptide compounds have an anti-thrombotic activity.

Described above are only preferred embodiments of the present disclosure and are not intended to limit the present disclosure. It should be understood that any modifications, replacements and improvements made by those skilled in the art without departing from the spirit and scope of the present disclosure should fall within the scope of the present disclosure defined by the appended claims.

What is claimed is:

1. An isaridin cyclodepsipeptide derivative of formula (I), or a pharmaceutically-acceptable salt thereof: 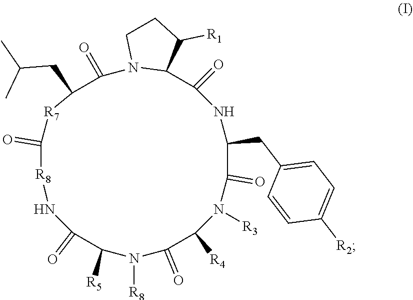
wherein R₁ is —H or —CH₃; R₂ is —H or —OH; R₃ and R₈ are each independently —H or —CH₃; R₄ and R₅ are each independently selected from the group consisting of —CH₃, —CH₂CH₃, —CH(CH₃)₂, —CH₂CH(CH₃)₂, —CH(CH₃)CH₂CH₃, —CH(OH)CH₃ and —CH₂Ar; R₆ is —CH₂CH₂—; and R₇ is —O—; and
the isaridin cyclodepsipeptide derivative is selected from the group consisting of:

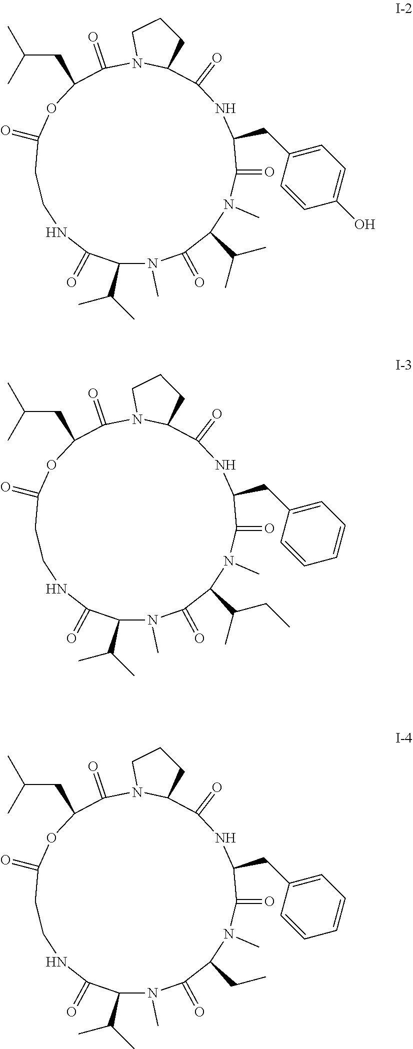 [SMILES: CC(C)C[C@@H]1OC(=O)CCNC(=O)[C@H](C(C)C)N(C)C(=O)[C@H](C(C)C)N(C)C(=O)[C@H](Cc2ccc(O)cc2)NC(=O)[C@@H]2CCCN2C1=O ; CCC(C)[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C ; CC[C@H]1C(=O)N(C)[C@@H](C(C)C)C(=O)NCCC(=O)O[C@@H](CC(C)C)C(=O)N2CCC[C@H]2C(=O)N[C@@H](Cc2ccccc2)C(=O)N1C]

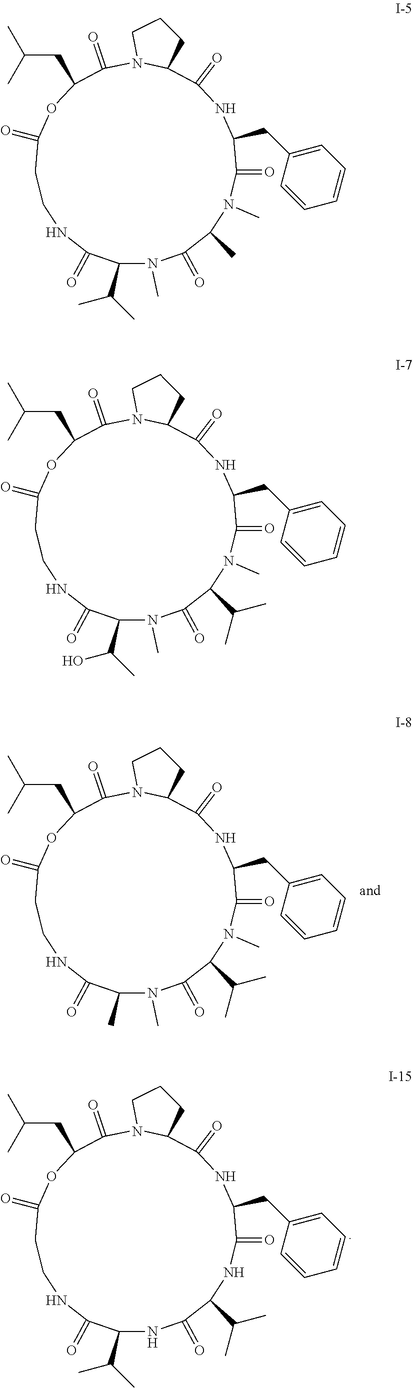 [SMILES: CC(C)C[C@@H]1OC(=O)CCNC(=O)[C@H](C(C)C)N(C)C(=O)[C@H](C)N(C)C(=O)[C@H](Cc2ccccc2)NC(=O)[C@@H]2CCCN2C1=O ; CC(C)C[C@@H]1OC(=O)CCNC(=O)[C@H](C(C)O)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C)N(C)C(=O)[C@H](C(C)C)N(C)C(=O)[C@H](Cc2ccccc2)NC(=O)[C@@H]2CCCN2C1=O ; CC(C)C[C@@H]1OC(=O)CCNC(=O)[C@H](C(C)C)NC(=O)[C@H](C(C)C)NC(=O)[C@H](Cc2ccccc2)NC(=O)[C@@H]2CCCN2C1=O]

2. A method for preparing the isaridin cyclodepsipeptide derivative of claim 1, comprising:
preparing the isaridin cyclodepsipeptide derivative from Beauveria felina SYSU-MS7908 by isolation and purification;
wherein a deposit number of the Beauveria felina SYSU-MS7908 is GDMCC No: 61059.

3. The method of claim 2, wherein the isaridin cyclodepsipeptide derivative is prepared through steps of:
(1) subjecting the Beauveria feline SYSU-MS7908 to enlarged culture to collect a fungal fermentation broth extract by an organic solvent; and
(2) subjecting the fungal fermentation broth extract to liquid separation extraction, concentration, silica gel column chromatography, Sephadex LH-20 gel column chromatography and reversed-phase high-performance liquid chromatography (RP-HPLC) to obtain the isaridin cyclodepsipeptide derivative.

4. The method of claim 3, wherein in step (1), the organic solvent is selected from the group consisting of acetone, ethyl acetate, methanol and ethanol.

5. The method of claim 3, wherein in step (2), the extraction is performed in ethyl acetate, chloroform or a combination thereof.

6. A method for treating inflammation in a subject in need thereof, comprising:
administering a therapeutically effective amount of the isaridin cyclodepsipeptide derivative of claim 1 or a pharmaceutically-acceptable salt thereof to the subject.

7. A method for treating thrombosis in a subject in need thereof, comprising:
administering a therapeutically effective amount of the isaridin cyclodepsipeptide derivative of claim 1 or a pharmaceutically-acceptable salt thereof to the subject.

8. A pharmaceutical composition, comprising:
the isaridin cyclodepsipeptide derivative of claim 1 or a pharmaceutically-acceptable salt thereof.
